(19) United States
(12) Patent Application Publication
(10) Pub. No.: US 2007/0293518 A1
(43) Pub. Date: Dec. 20, 2007

(54) PROLONGED IMPROVEMENT OF RENAL FUNCTION COMPRISING INFREQUENT ADMINISTRATION OF AN AA1RA

(76) Inventors: Howard Dittrich, San Diego, CA (US); Brian Farmer, San Diego, CA (US); Randy Woods, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/764,018

(22) Filed: Jun. 15, 2007

Related U.S. Application Data
(60) Provisional application No. 60/814,232, filed on Jun. 16, 2006.

(51) Int. Cl. A61K 31/522

(52) U.S. Cl. 514/263.32; 514/263.34

(57) ABSTRACT

A method for treating a patient, comprising providing a therapeutically-effective amount of an AA₁RA to the patient; and informing the patient or a medical care worker that administration of the AA₁RA can provide an improvement in renal function that persists for a time period following administration of the AA₁RA, and wherein the time period is at least 3 days.

### RELATED APPLICATIONS

The present application claims priority to U.S. Provisional Application Ser. No. 60/814,232, filed on Jun. 16, 2006, by Dittrich et al., and entitled “PROLONGED IMPROVEMENT OF RENAL FUNCTION COMPRISING INFREQUENT ADMINISTRATION OF AN AA₁RA,” the entire disclosure of which is herein incorporated by reference in its entirety.

### BACKGROUND OF THE INVENTION

1. Field of the Invention

The embodiments disclosed herein relate to methods of prolonged improvement of renal function in individuals in need of maintenance, restoration, or improvement in renal function.

2. Description of the Related Art

Physicians are becoming increasingly aware that an important relationship exists between the heart and the kidney in congestive heart failure (CHF). This complex interaction is known as the cardio-renal syndrome. Multiple studies have demonstrated that renal dysfunction is a strong independent predictor of worse short- and long-term outcomes in patients with CHF. For example during hospital stays, a greater than 0.3 milligram per deciliter increase in serum creatinine, a commonly used marker of worsening renal function, predicts longer hospital stays, higher heart failure readmission rate and higher mortality rates. Worsening renal function makes effective treatment with standard diuretics more difficult, may increase workload of the heart, trigger arrhythmias and result in the production of chemicals that may adversely affect the cardiovascular system. In addition, in the presence of renal impairment, physicians often decrease use of outcome-improving drugs such as ACE inhibitors for fear that these drugs will further worsen renal function. Currently, there is a need for effective therapies to improve renal function, particularly in individuals with heart failure.

### SUMMARY OF THE INVENTION

In some embodiments, provided herein are methods for treating a patient including the steps of providing a therapeutically-effective amount of an AA₁RA to the patient; and informing the patient or a medical care worker that administration of the AA₁RA can provide an improvement in renal function that persists for a time period following administration of the AA₁RA, and wherein the time period is at least about 3 days, at least about 5 days, at least about 7 days, at least about 10 days, at least about 14 days or more.

Other embodiments provided herein relate to methods for treating a patient that include the steps of identifying a patient in need of a relatively long term improvement in renal function and administering a relatively shorter term AA₁RA therapy to the patient. In some embodiments, the relatively long term improvement is an improvement that persists at least about 3 days after the AA₁RA therapy. For example, the improvement persists at least about 5 days after the AA₁RA therapy, at least about 7 days after the AA₁RA therapy, at least about 10 days after the AA₁RA therapy, or at least about 14 days after the AA₁RA therapy or more. In some embodiments, the methods include the step of informing the patient or a medical care worker that improvement in renal function persists for a time period following administration of the AA₁RA, and wherein the time period is at least about 3 days, at least about 5 days, at least about 7 days, at least about 10 days, at least about 14 days, or more, after the AA₁RA therapy.

In some embodiments, the patient is suffering from congestive heart failure. In some embodiments, the AA₁RA can one of the following: KW-3902, BG-9719, BG-9928, CVT-124, or SLV-320, or a pharmaceutically acceptable salt, ester, amide, prodrug, or metabolite thereof.

In some embodiments, the therapeutically effective amount of KW-3902 can be between about 2.5 to about 70 mg/dose, for example, between about a 20 mg/dose to about 50 mg/dose. In preferred embodiments, the therapeutically effective amount of KW-3902 can be about 30 mg/dose.

Other embodiments relate to methods for treating a patient having impaired renal function, that include the steps of designating a course of therapy that is intended to achieve improved renal function for at least a predetermined period, wherein the course of therapy includes administration of an AA₁RA, and administering the course of therapy to the patient, wherein the AA₁RA administration is completed at least about 3 days prior to completion of the predetermined period.

Yet other embodiments provide methods of improving and/or maintaining renal function in individuals with stable congestive heart failure (CHF) taking chronic diuretics, including the steps of administering to the individual a therapeutically effective amount of an adenosine AA₁RA in about four day to about monthly intervals, wherein the individual simultaneously continues said chronic diuretic therapy throughout the course of treatment with said AA₁RA.

Still other embodiments relate to methods for treating individuals experiencing mild renal impairment who undergoing diuretic therapy, including the steps of administering to the individual a therapeutically effective amount of an AA₁RA on a bi-weekly to monthly basis.

Yet other embodiments relate to methods for slowing or reversing an existing or developing renal impairment in a patient including the steps of administering to the patient an effective periodic dose of an AA₁RA between about once every four days and about once every month.

In some embodiments, the AA₁RA can be KW-3902, BG-9719, BG-9928, CVT-124, or SLV-320, or a pharmaceutically acceptable salt, ester, amide, prodrug, or metabolite thereof. In preferred embodiments, the AA₁RA is KW-3902 or a pharmaceutically acceptable salt, ester, amide, prodrug, or metabolite thereof.

In some embodiments, the therapeutically effective amount of KW-3902 can be between about 2.5 to about 70 mg/dose, for example, between about a 20 mg/dose to about 50 mg/dose. In preferred embodiments, the therapeutically effective amount of KW-3902 can be about 30 mg/dose.

In some embodiments, the AA₁RA can be administered in approximately 4 to 30 day intervals, for example in 7 to 30 day intervals, for example 7 to 9 day intervals, etc. IN some embodiments, the AA₁RA can be administered in approximately 14 day intervals.

Some embodiments relate to the use of an AA₁RA in the preparation of a medicament for treating renal dysfunction, for use by a patient in need of a persistent improvement in renal function that persists for at least 3 days following administration of the AA₁RA.

Other embodiments relate to the use of a therapeutically effective amount of an AA₁RA in the preparation of a medicament for treating renal dysfunction in a patient in need of a relatively long term improvement in renal function, wherein the medicament is administered over a relatively shorter term to said patient.

Yet other embodiments relate to the use of a therapeutically effective amount of an AA₁RA in the manufacture of a medicament for use in a course of therapy to treat renal function in a patient over a predetermined period of time, wherein the administration of the medicament is completed at least 3 days prior to completion of the predetermined period.

Other embodiments relate to the use of a therapeutically effective amount of an AA₁RA in the manufacture of a medicament for improving and/or maintaining renal function in individuals with stable congestive heart failure (CHF) taking chronic diuretics, for administration to said individual at intervals of four to thirty days, wherein said individual continues chronic diuretic therapy throughout the course of AA₁RA therapy.

Still other embodiments relate to the use of a therapeutically effective amount of an AA₁RA in the manufacture of a medicament for treating an individual experiencing mild renal impairment, wherein said individual is undergoing diuretic therapy, for administration on a bi-weekly to a monthly basis.

Use of an effective periodic amount of an AA₁RA in the manufacture of a medicament for slowing or reversing an existing or developing renal impairment in a patient, for administration at intervals of between four days and thirty days.

### BRIEF DESCRIPTION OF THE DRAWINGS

FIGS. 1A and 1B show the simulated serum concentrations of KW-3902 and its metabolite, M1-trans, over time following single 25 mg or 30 mg intravenous doses of KW-3902. The KW-3902 is administered over 2, 4, 6, 8, 12, or 24 hours, as indicated in the figure legend. Also shown in FIG. 1A is the simulated serum concentration of KW-3902 and M1-trans following a 10 mg dose of KW-3902 administered over two hours, followed by a 15 mg dose of KW-3902 over 4 hours (10/15), or a 15 mg dose of KW-3902 over two hours, followed by a 10 mg dose of KW-3902 over 4 hours (15/10). Also shown in FIG. 1B is the simulated serum concentration of KW-3902 and M1-trans following a 10 mg dose of KW-3902 over 2 hours followed by a 20 mg dose of KW-3902 over 4 hours (10/20); a 15 mg dose of KW-3902 administered over 2 hours followed by a 15 mg dose of KW-3902 over 4 hours (15/15), or a 20 mg dose of KW-3902 administered over 2 hours followed by a 10 mg dose of KW-3902 over 4 hours (20/10). The arrows in FIG. 1B indicate the simulated serum concentration of KW-3902 and M1-trans following a single dose of 25 mg or 30 mg KW-3902 administered over the course of 4 hours.

FIG. 2 shows the percent change in glomerular filtration rate (mL/min) over baseline in subjects 0-2, 2-4, and 4-8 hours after treatment with 30 mg KW-3902 or placebo.

FIG. 3 shows the change in renal plasma flow over baseline in subjects 0-2, 2-4, and 4-8 hours after treatment with KW-3902 or placebo.

FIG. 4 shows the change in creatinine clearance rate compared to baseline (mL/min) 0-3 hours, 3-6 hours, 6-9 hours, and 9-12 hours after treatment with placebo, 10 mg KW-3902, 30 mgKW-3902 or 60 mg KW-3902.

FIG. 5 shows the change in glomerular filtration rate (mL/min) over baseline in subjects 4 to 8 hours after treatment in a double crossover study with 30 mg KW-3902 or placebo, as described in Example 1.

FIG. 6 shows the % incidence of sustained worsening renal function at the last observation, or at 7 days, following treatment with placebo, 2.5mg KW-3902, 15 mg KW-3902, 30 mg KW-3902, or 60 mg KW-3902.

FIG. 7 shows the percentage of subjects with acute CHF treated with 10 mg, 20 mg, or 30 mg KW-3902 or placebo exhibiting worsening renal function over the indicated time periods following treatment, as described in Example 1.

FIG. 8 shows the change in mean serum creatinine levels over the indicated time period in subjects with acute CHF and mild to severe renal impairment requiring intravenous diuretic therapy following treatment with 10 mg, 20 mg, or 30 mg KW-3902, or placebo as described in Example 1.

### DETAILED DESCRIPTION OF THE PREFERRED EMBODIMENT

The present disclosure is based, in part, on the surprising discovery that adenosine Al receptor antagonists provide a persistent beneficial effect on renal function which lasts beyond the time period that the AA₁RA or its metabolites persist in the bloodstream. In studies involving patients with stable or acute congestive heart failure and impaired renal function, we found AA₁RA treatment resulted in an improvement in renal function that lasted well beyond the time after which the AA₁RA was cleared by the patient.

Adenosine is a naturally occurring molecule that acts via several kinds of adenosine receptors throughout the body. Adenosine plays an important role in regulating renal function. In a healthy kidney, adenosine fine-tunes blood flow, renal function and the handling of salt and water. The kidney produces adenosine constitutively to regulate glomerular filtration and electrolyte reabsorption mediated by the adenosine A₁-receptor system. In the kidney, Adenosine Al receptors govern the vasoconstriction response of the afferent (preglomerular) renal arteriole. Adenosine and other adenosine agonists cause a reduction in the blood flow to the kidney, and thus a reduction in the glomerular filtration rate.

Renal function can be impaired in subjects with fluid overload and/or congestive heart failure (CHF). Therapeutics such as diuretics, ACE inhibitors, and beta-blockers are commonly administered to patients with fluid overload and/or congestive heart failure. However, these compounds may further impair renal function. Individual diuretics act on a specific segment of nephrons, e.g., the proximal tubule, loop of Henle, or distal tubule. Thus, for example, a loop diuretic inhibits re-absorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium re-absorption elsewhere in the nephron; the other is to feedback via adenosine Al receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

Provided herein are methods of treating patients with an AA₁RA. As used herein, the term AA₁RA refers to compounds that selectively antagonize the adenosine Al receptor. The scope of the present invention includes all those AA₁RAs now known and all those AA₁RAs to be discovered in the future. Exemplary AA₁RAs useful in the embodiments described herein include, but are not limited to KW-3902, BG-9719, BG-9228, CVT-124, SLV-320 or pharmaceutically acceptable salts, esters, amides, prodrugs, or metabolites thereof. It will be appreciated that the terms “KW-3902,” “BG-9719,” “BG-9228,” “CVT-124,” and “SLV-320” is meant to refer to KW-3902, BG-9719, BG-9228, CVT-124, SLV-320 as well as any pharmaceutically acceptable salt, ester, amide, metabolite, or prodrug thereof, respectively. BG-9719 is described in U.S. Pat. No. 5,446,046, U.S. Pat. No. 5,631,260, and U.S. Pat. No. 5,668,139, the disclosures of which are each herein incorporated by reference in its entirety. In some embodiments, the AA₁RA can be (3-[4-(2,6-Dioxo-1,3-dipropyl-2,3,6,7-tetrahydro-1H-purin-8-yl)-bicyclo[2.2.2]oct-1-yl]-propionic acid (BG-998).

KW-3902 is a xanthine-derived adenosine A₁ receptor antagonist (AA₁RA). Its chemical name is 8-(3-noradamantyl)-1,3-dipropylxanthine, also known as 3,7-dihydro-1,3-dipropyl-8-(3-tricyclo[3.3.1.0^{3,7} nonyl)-1H-purine-2,6-dione, and its structure

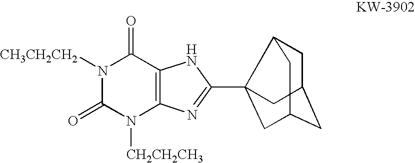

KW-3902 also blocks the TGF mechanism mediated by adenosine (via A₁ receptors) described above. This ultimately allows for increased GFR and improved renal function, which ultimately results in more fluid passing through the loop of Henle and the distal tubule. In addition, KW-3902 inhibits the reabsorption of sodium (and, therefore, water) in the proximal tubule, which results in diuresis. Furthermore, KW-3902 is an inhibitor of TGF, which can counteract the adverse effect of some diuretics, such as proximal diuretics, which activate or promote TGF.

KW-3902 and related compounds useful in the practice of the present invention are described, for example, in U.S. Pat. Nos. 5,290,782, 5,395,836, 5,736,528, 6,210,687, and 6,254,889, the entire disclosure of all of which are hereby incorporated by reference herein, including any drawings.

Other AA₁RA's useful in the present invention are those disclosed in, for example, U.S. Pat. No. 4,769,337, U.S. Pat. No. 5,746,046, U.S. Pat. No. 6,818,647, U.S. Pat. No. 6,605,600, U.S. Pat. No. 6,649600, U.S. Patent Publication No. 2005/0245546, U.S. Patent Publication No. 2004/0259889, U.S. Patent Publication No. 2002/0115687, and U.S. Pat. No. 7,022,686, the disclosures of which are each hereby expressly incorporated by reference in their entireties, including all drawings.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Any amine, hydroxy, or carboxyl side chain on the metabolites, esters, or amides of the compounds disclosed herein can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

The term “metabolite” refers to a compound to which the starting compound is converted within the cells of a mammal. For example, a metabolite of KW-3902 refers to a compound to which KW-3902 is converted within the cells of a mammal. The pharmaceutical compositions of the present invention may include a metabolite of KW-3902 instead of KW-3902. The scope of the methods described herein includes those instances where a compound, e.g., KW-3902, is administered to the patient, yet the metabolite is the bioactive entity.

Several metabolites of KW-3902 are known and are useful in the methods disclosed herein. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a ═O group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), 8-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine. KW-3902 metabolites used in the pharmaceutical compositions or methods disclosed herein may be a xanthine-derivative compound. The xanthine-derivative compound may be a compound of Formula I or a pharmaceutically acceptable salt thereof, 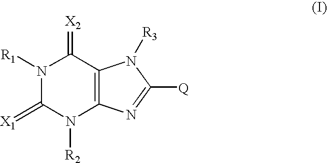
where
each of X₁ and X₂ independently represents oxygen or sulfur;
Q represents: 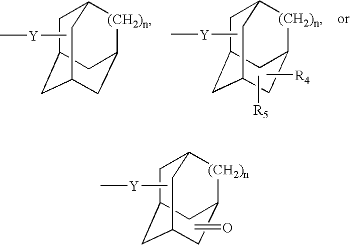
   where Y represents a single bond or alkylene having 1 to 4 carbon atoms, n represents 0 or 1;

each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or

R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 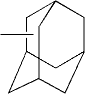
then R₁, R₂ and R₃ are not simultaneously methyl.

In some embodiments, both of R₁ and R₂ of the compound of Formula I are lower alkyl and R₃ is hydrogen; and both of X₁ and X₂ are oxygen. In other embodiments, R₁, R₂ and R₃ independently represent hydrogen or lower alkyl. In still other embodiments, each of R₁ and R₂ independently represents allyl or propargyl and R₃ represents hydrogen or lower alkyl. In certain embodiments, X₁ and X₂ are both oxygen and n is 0.

In some embodiments, R₁ is hydroxy-substituted, oxo-substituted or unsubstituted propyl; R₂ is hydroxy-substituted or unsubstituted propyl; and Y is a single bond. In other embodiments, R₁ is propyl, 2-hydroxypropyl, 2-oxopropyl or 3-oxopropyl; R₂ is propyl, 2-hydroxypropyl or 3-hydroxypropyl.

In some embodiments Q is 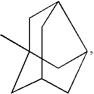
while in other embodiments Q is 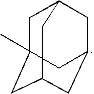
In other embodiments, Q is 9-hydroxy, 9-oxo or 6-hydroxy substituted 3tricyclo[3.3.1.0^{3,7}]nonyl, or 3-hydroxy-1tricyclo [3.3.1.1^{3,7}]decyl.

In certain embodiments, the KW-3902 metabolite is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine, 8-(cis-9-hydroxy-3-tricyclo[3.3.1.0^{3,7}]nonyl)-1,3-dipropylxanthine, 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine, or a pharmaceutically acceptable salt thereof.

In other embodiments, the xanthine derivative is a xanthine epoxide-derivative compound of Formula II or Formula III, or a pharmaceutically acceptable salt thereof, 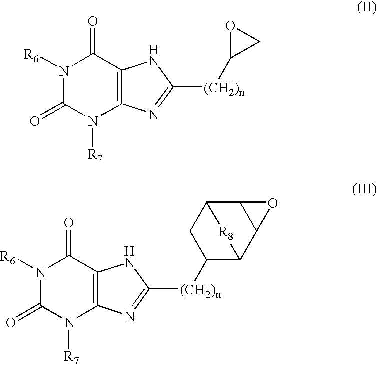
where R₆ and R₇ are the same or different, and can be hydrogen or an alkyl group of 1-4 carbons, R₈ is either oxygen or (CH₂)₁₋₄, and n=0-4.

The xanthine epoxide-derivative compound may be

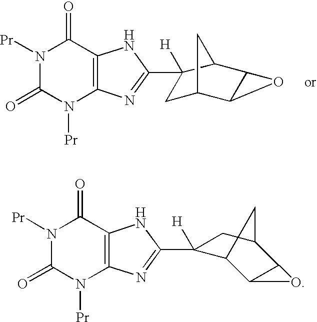

In some methods, the subject is provided a therapeutically effective amount of an AA₁RA. The term “therapeutically effective amount” as used herein refers to that amount of a composition being administered which will relieve to some extent one or more of the signs or symptoms of the disorder being treated. For example, the patient can be administered an amount of an AA₁RA effective to improve or maintain renal function. Renal function refers to the ability the kidney to excrete waste and maintain a proper chemical balance. Renal function can be measured by plasma concentrations of creatinine, urea, and electrolytes. Creatinine is a byproduct of normal muscle metabolism that is produced at a fairly constant rate in the body and normally filtered by the kidneys and excreted in the urine. It will be appreciated that any method known to those skilled in the art for measuring renal function can be used in the methods described herein. In some embodiments, serum creatinine levels, urine creatinine levels, glomerular filtration rate (GFR) and renal plasma flow (RPF) are be used to assess renal function.

In the context of the present disclosure, by “maintaining” renal function it is meant that the renal function, as measured by the methods described herein, remains unchanged for a period of time after the start of the therapy. In other words, the phrase “maintaining” renal function refers to a slowing or arresting of the rate of renal impairment, as measured for example, by urine creatinine clearance, serum creatinine levels, GFR, RPF, and the like.

In the context of the present disclosure, by “slowing” renal impairment it is meant that a decrease in renal function, as manifested for example by a decreasing creatinine clearance rate, is slowed or arrested for a period of time after the start of the therapy. In other words, by “slowing” renal impairment it is meant that the rate of renal impairment, e.g., the rate of decrease in the urine creatinine clearance rate, the rate of increase in serum creatinine levels, the rate of decrease in the glomerular filtration rate, or the rate of decrease in the renal plasma flow, is slowed or arrested for a period of time after the start of therapy.

In the context of the present disclosure, by “improving” renal function can refer to a reversal of an existing renal impairment, or a restoration of renal function following renal impairment. In some embodiments, an improvement in renal function refers to a slowing or arresting of worsening renal impairment. Improvements in renal function can be manifested for example by a cessation in a decreasing rate of creatine clearance, a cessation in a decrease in GFR or RPF, or an increase in the rate of urine creatinine clearance, an increase in the GFR, or an increase in RPF for a period of time after the start of therapy.

In some embodiments, the maintenance or improvement of renal function persists for a period of time of at least 1 hour, at least 2 hours, at least 4 hours, at least 6 hours, at least 8 hours, at least 10 hours, at least 12 hours, at least 14 hours, at least 16 hours, at least 20 hours, at least 22 hours, at least 24 hours, at least 36 hours, at least 48 hours, at least 60 hours, at least 72 hours, at least 84 hours, at least 96 hours, at least 108 hours, at least 120 hours, at least 132 hours, at least 144 hours, at least 156 hours, at least 168 hours, at least 180 hours, at least 16 days, at lest 17 days at least 18 days, at least 19 days, at least 20 days, at least 21 days, at least 30 days, or more, or any amount of time in between. Preferably, the maintenance or improvement in renal function persists for at least about 3 days or more.

In some embodiments, the methods described herein provide long-term improvement or maintenance of renal function. As used herein, the phrases “long term maintenance of renal function” or “long-term improvement in renal function” refer to maintenance or improvement in renal function that persists for a period of time of at least 1 hour, at least 2 hours, at least 4 hours, at least 6 hours, at least 8 hours, at least 10 hours, at least 12 hours, at least 14 hours, at least 16 hours, at least 20 hours, at least 22 hours, at least 24 hours, at least 36 hours, at least 48 hours, at least 60 hours, at least 72 hours, at least 84 hours, at least 96 hours, at least 108 hours, at least 120 hours, at least 132 hours, at least 144 hours, at least 156 hours, at least 168 hours, at least 180 hours, at least 16 days, at lest 17 days at least 18 days, at least 19 days, at least 20 days, at least 21 days, at least 30 days, or more, or any amount of time in between. Preferably, long term maintenance or long term improvement in renal function persists for at least about 3 days or more.

In some embodiments, the patient is administered a therapeutically effective amount of an AA₁RA. The term “effective amount” or “therapeutically effective amount” means an amount effective, when administered to a patient, to provide any therapeutic benefit. A therapeutic benefit may be an amelioration of symptoms, e.g., an amount effective to maintain or improve renal function. In certain circumstances a patient may not present symptoms of a condition for which the patient is being treated.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patients overall feeling of well being or appearance. Treatment may also include lengthening the life of the patient, even if the symptoms are not alleviated, the disease conditions are not ameliorated, or the patient's overall feeling of well being is not improved. Thus, in the context of the present invention, increasing the urine output volume, decreasing the level of serum creatinine, or increasing creatinine clearance, may be considered treatment, even if the patient is not cured or does not generally feel better

Thus, in some embodiments, the individual is administered an amount of an AA₁RA effective to cause an increase in urine creatinine clearance of about 0.01 mg/mL, 0.02 mg/mL, 0.03 mg/mL; 0.04 mg/mL, 0.05 mg/mL, 0.06 mg/mL, 0.07 mg/mL, 0.08 mg/mL, 0.09 mg/mL, 0.10 mg/mL, 0.11 mg/mL, 0.12 mg/mL, 0.13 mg/mL, 0.14 mg/mL, 0.15 mg/mL, 0.16 mg/mL, 0.17 mg/mL, 0.1 8 mg/mL, 0.19 mg/mL, 0.20 mg/mL or more. In some embodiments, the increase in creatinine clearance rate persists over at least 3 days, at least 4 days, at least 5 days, at least 6 days, at least 7 days, at lest 8 days, at least 9 days, at least 10 days, at least 12 days, at least 14 days, at least 16 days, at least 18 days, at least 20 days, at least 25 days, or at least 30 days, or any amount of time in between. In some embodiments, the increase in creatinine clearance rate persists for more than 30 days.

Accordingly, in some embodiments the patient is administered an AA₁RA, (e.g., KW-3902) in a dose of at least about 0.5 mg, 1.0 mg, 1.5 mg, 2.0 mg, 2.5 mg, 3 mg, 4 mg, 5 mg, 10 mg, 15 mg, 20 mg, 25 mg, 30 mg, 35 mg, 40 mg, 45 mg, 50 mg, 55 mg, 60 mg, 70 mg, 80 mg, 90 mg, 100 mg, or any number in between. In some embodiments, the AA₁RA is administered in a dose lower than 0.5 mg. In other embodiments, the AA₁RA is administered in a dose higher than 100 mg.

In some embodiments, the method can include the step of informing the patient or medical care worker that administration of the AA₁RA can maintain or improve renal function that persists for a time period following administration of the AA₁RA. For example, the patient or medical care worker can be informed renal function can be maintained or improved for at least 1 hour, at least 2 hours, at least 4 hours, at least 6 hours, at least 8 hours, at least 10 hours, at least 12 hours, at least 14 hours, at least 16 hours, at least 20 hours, at least 22 hours, at least 24 hours, at least 36 hours, at least 48 hours, at least 60 hours, at least 72 hours, at least 84 hours, at least 96 hours, at least 108 hours, at least 120 hours, at least 132 hours, at least 144 hours, at least 156 hours, at least 168 hours, at least 180 hours, at least 16 days, at lest 17 days at least 18 days, at least 19 days, at least 20 days, at least 21 days, at least 30 days, or more, or any amount of time in between.

As used herein, the term “informing” means referring to or providing, published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

A “medical care worker” means a worker in the health care field who may need or utilize information regarding an active agent, including a dosage form thereof, including information on safety, efficacy, dosing, administration, or pharmacokinetics. Examples of medical workers include physicians, pharmacists, physician's assistants, nurses, aides, caretakers (which can include family members or guardians), emergency medical workers, and veterinarians.

In some embodiments, the methods include the step of designating a course of therapy that is intended to achieve improved renal function for at least a predetermined period, wherein the course of therapy includes administration of an AA1RA. For example, in some embodiments, the course of therapy can include the administration of an AA1RA with one or more therapeutics, such as non adenosine-modifying diuretics, ACE inhibitors, ARBs, beta blockers, aldosterone inhibitors and the like, as discussed below. In some embodiments, the course of therapy can be at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 25 days, at least about 30 days, at least about 35 days, at least about 40 days, at least about 45 days, at least about 50 days, at least about 60 days, or more, or any amount of time in between. In some embodiments, the AA₁RA administration can be completed at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 25 days, at least about 30 days, at least about 35 days, at least about 40 days, at least about 45 days, at least about 50 days, at least about 60 days, or more prior to completion of the predetermined period for the course of the therapy.

In certain embodiments, the individual being treated by the methods of the present invention suffers from renal impairment. In other embodiments, the individual does not suffer from renal impairment. It will be appreciated that any method known to those skilled in the art for measuring renal function can be used in the methods described herein. For example, serum creatinine levels, creatinine clearance, glomerular filtration rate (GFR) and renal plasma flow (RPF) can be used to assess renal function. For example, individuals suffering from renal impairment can exhibit a creatinine clearance rate of about 20 mL/min to about 80 mL/min, e.g., about 25 mL/min, about 30 mL/min, about 35 mL/min, about 40 mL/min, about 45 mL/min, about 50 mL/min, about 55 mL/min, about 60 mL/min, about 65 mL/min, about 70 mL/min, about 75 mL/min, about 80 mL/min, or more, or any number in between. In some embodiments, patients exhibit a GFR of less than about 80 mL/min, for example about 20 mL/min, 30 mL/min, 40 mL/min, 50 mL/min, 60 mL/min 70 mL/min or 75 mL/min, or any number in between.

Accordingly, in some embodiments, the patient exhibits mildly impaired renal function (e.g., a GFR of about 50 to about 80 mL/min). In some embodiments, the patient exhibits moderately impaired renal function (e.g., a GFR of about 30 mL/,min to about 50 mL/min). In yet other embodiments, the patient exhibits severely impaired renal function (e.g, a GFR of about 0 mL/min to about 30 mL/min). Individuals with impaired renal function can include individuals who suffer from heart failure, such as congestive heart failure, or other maladies that result in fluid overload, without having yet disrupted normal kidney function.

In some embodiments, the individual being treated by the methods disclosed herein have congestive heart failure. Congestive heart failure (CHF) is a condition in which impaired heart function exists. The impaired heart function can be accompanied by a build-up of body fluid. CHF often occurs when cardiac output is insufficient to meet metabolic demands of the body, or when the heart cannot meet the demands of operating at increased levels of filling/diastolic pressure. In some embodiments, the individual being treated by the methods disclosed herein have stable congestive heart failure. As used herein, the term “stable congestive heart failure” or “chronic congestive heart-failure” is given its ordinary meaning. For example, an individual with stable or chronic congestive heart failure can refer to an individual who has a documented history of congestive heart failure, including at least one prior symptom. Non-limiting examples of symptoms of congestive heart failure include dyspnea on exertion or at rest, orthopnea, paroxysmal nocturnal dyspnea, abdominal swelling, and peripheral edema. Stable congestive heart failure can also describe individuals with one prior sign of heart failure. Non-limiting examples of signs of congestive heart failure include jugular venous distension, ventricular gallop, rales, hepatomegaly, ascites or peripheral edema. Stable congestive heart failure can further refer to a current absence of excessive congestion, e.g., no or little ascites, and only mild basilar pulmonary rales and peripheral edema.

In some embodiments, individuals identified in the methods provided herein have acute congestive heart failure. Patients presenting with acute decompensated CHF can have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries can result in an acute rise in the left ventricular (LV) filing pressures. The rise in the LV filing pressures results in pulmonary edema and dyspnea. In some cases the acute CHF is due to an acute increase in systemic vascular resistance or volume overload secondary to medication non-compliance or dietary indiscretion.

In some embodiments, the individual being treated by the methods disclosed herein have acute fluid overload. In some embodiments, the individual has CHF and acute fluid overload. In other embodiments, the individual does not have CHF, but has acute fluid overload. In some embodiments, the patients presenting with acute fluid overload are in need of intravenous diuretic treatment. In some embodiments, individuals with acute fluid overload are in need of short-term hospitalization, and/or in need of intravenous diuretic therapy to treat the fluid overload. Patients with acute fluid overload can be identified using standard clinical diagnostic procedures. Non-limiting factors that are commonly evaluated in determining whether an individual requires hospitalization for acute fluid overload include pitting edema (2+) of lower extremities; jugular venous distension; pulmonary edema or pleural effusion; ascites; paroxysmal nocturnal dyspnea or 2-pillow orthopnea.

In some embodiments the subjects are in need of intravenous diuretic therapy. Patients in need of intravenous diuretic treatment can be identified using conventional diagnostic methods. For example, an individual in need of IV diuretic treatment can refer to an individual exhibiting one or more signs or symptoms of CHF, e.g., congestion of the lungs, liver, intestines and peripheral compartments, shortness of breath (dyspnea) fatigue, orthopnea, rales, pitting edema, elevated central venous pressure, pulmonary congestion, weight gain, volume overload, and elevated filling pressures.

In some embodiments, the patients are receiving chronic diuretic therapy. As used herein, the phrase “chronic diuretic therapy” or variations thereof e.g., “chronic diuretics” refers to continuous diuretic therapy (e.g., at least daily therapy) for a period of time. Individuals identified as receiving chronic diuretic therapy, therefore, can refer to individuals that have been taking daily diuretics continuously over at least about three weeks, at least about 4 weeks, at least about 6 weeks, at least about 10 weeks, or at least about 12 weeks, or more, or for any period of time in between. Continuation of chronic diuretic therapy refers to substantially uninterrupted daily diuretic therapy. In some embodiments, the patient continues chronic diuretic therapy throughout the course of treatment with the AA₁RA.

Combinations of AA₁RAs with Other Therapeutics

In some embodiments provided herein, the subject to be treated by the methods described herein can be administered an AA₁RA, in combination with another compound or therapeutic such as a non adenosine-modifying diuretic, an ACE, an ARB, a beta blocker, an aldosterone inhibitor or other compound or any combination thereof.

Diuretics are classified based on their mode and locus of action. In some embodiments, the non-adenosine modifying diuretic is a proximal diuretic, i.e., a diuretic that principally acts on the proximal tubule, Exemplary proximal diuretics include carbonic anhydrase inhibitors such as acetazolamide methazolamide, and dichlorphenamide. Any proximal diuretic now known or later discovered is within the scope of the embodiments disclosed herein. Loop diuretics principally act on the loop of Henle. Examples of loop diuretics useful in the methods described herein include, but are not limited to, furosemide (LASIX®), bumetanide (BUMEX®), and torsemide (TOREM®). Any loop diuretic now known or later discovered is within the scope of the embodiments disclosed herein. Distal diuretics that principally act on the distal nephron. Examples of distal diuretics useful in the methods described herein include, but are not limited to, metolazone, thiazides and amiloride. any distal diuretic now known or discovered in the future is within the scope of the embodiments disclosed herein.

In some embodiments, the subject can receive a beta-blocker in addition to AA₁RA therapy. A number of beta-blockers are commercially available. These compounds include, but are not limited to, acebutolol hydrochloride, atenolol, betaxolol hydrochloride, bisoprolol fumarate, carteolol hydrochloride, esmolol hydrochloride, metoprolol, metoprolol tartrate, nadolol, penbutolol sulfate, pindolol, propranolol hydrochloride, succinate, and timolol maleate. Beta-blockers, generally, are beta₁ and/or beta₂ adrenergic receptor blocking agents, which decrease the positive chronotropic, positive inotropic, bronchodilator, and vasodilator responses caused by beta-adrenergic receptor agonists. The embodiments described herein include all beta-blockers now known and all beta-blockers discovered in the future.

In some embodiments, the patient can receive an angiotensin converting enzyme inhibitor or an angiotensin II receptor blocker in addition to AA₁RA therapy. A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. The embodiments described herein include all ACE inhibitors now known and all ACE inhibitors discovered in the future.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs function stems from their ability to block the binding of angiotensin II, which would normally cause vessels to constrict. The embodiments disclosed herein include all ARBs now known and all ARBs discovered in the future.

In some embodiments, the patient can receive an aldosterone inhibitor in addition to AA₁RA therapy. A number of aldosterone inhibitors are commercially available. These compounds include, but are not limited to, spironolactone (ALDACTONE®) and eplerenone (INSPRA®). The embodiments disclosed herein include all aldosterone inhibitors now known and all aldosterone inhibitors discovered in the future.

In some embodiments, the patient can receive a prophylactically or therapeutically effective amount of an anticonvulsant in addition to AA₁RA therapy. Several anticonvulsants are known in the art and are useful in the compositions and methods described herein. See, e.g., U.S. Patent Application Publication No. 2005/0070524 Extensive listings of anticonvulsants can also be found, e.g., in Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083, the disclosures of which are hereby expressly incorporated by reference in their entireties. Non-limiting examples of anticonvulsants that can be used in the compositions and methods disclosed hererin include diazepam, midazolam, phenytoin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof. However, the inclusion of other anticonvulsants, now known or discovered in the future, is within the scope of the present invention.

In some embodiments, an anticonvulsant can be provided in such amount as will be therapeutically or prophylactically effective in the treatment or control of seizures. It will be appreciated that the amount of anticonvulsant contained in an individual dose of each dosage form of the compositions need not in itself constitute an effective prophylactic amount, as the necessary effective amount could be reached by administration of a number of individual doses. Those skilled in the art will appreciate that the amount of anticonvulsant agent present in the compositions and administered to individuals disclosed herein will vary depending upon the age, sex, and bodyweight of the subject to be treated, the particular method and scheduling of administration, and what other anticonvulsant agent, if any is present in the compositions disclosed herein or administered in the methods disclosed herein. Dosage amounts for an individual patient may thus be above or below the typical dosage ranges. Generally speaking, the anticonvulsant agent can be employed in any amount known to be effective at treating, preventing or controlling seizures. The doses may be single doses or multiple doses per day, with the number of doses taken per day and the time allowed between doses varying depending on the individual needs of the patient. Optimization of treatment, including dosage amount, method and time of administration can be routinely determined by the skilled practitioner. Specific dosage levels for anticonvulsants that can be used in the pharmaceutical compositions and methods described herein, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of which are all hereby expressly incorporated by reference.

Administration of KW-3902 and Other Therapeutics

The embodiments described herein include the administration of an AA1RA to a patient. In some embodiments, the AA₁RA, e.g., KW-3902 or a pharmaceutically acceptable salt, ester, amide, metabolite, or prodrug thereof, can be administered parenterally. For example, intramuscularlly, subcutaneously, intravenously, via intramedullary injections, as well as intrathecal, direct intraventricular, intraperitoneal, intranasal, or intraocular injections or the like. Preferably, the AA₁RA, e.g., KW-3902 or a pharmaceutically acceptable salt, ester, amide, metabolite, or prodrug thereof can provided intravenously in a continuous infusion.

In some embodiments, an AA₁RA, e.g., KW-3902 is provided in a single dose during the administration. For example, in some embodiments, about 2.5 mg. 5 mg, 10 mg, 15 mg, 20 mg, 30 mg, 40 mg, 50 mg, 60 mg, 80 mg, 100 mg or more of an AA₁RA, e.g., KW-3902 can be provided in a single dose, for example in a continuous intravenous infusion. In some embodiments, an AA₁RA, eg., KW-3902 is provided in more than one dose during the administration, for example, two, three or more doses of an AA₁RA, e.g, KW-3902 can be provided in a single continuous intravenous infusion. Accordingly, in some embodiments, a dose of about 10 mg of an AA₁RA, e.g., KW-3902 can be provided followed by a dose of about 15 mg or 20 mg in a continuous infusion. Alternatively, a dose of about 15 mg or 20 mg of an AA₁RA, e.g., KW-3902 or a pharmaceutically acceptable salt, ester, amide, metabolite, or prodrug thereof can be provided, followed by a dose of about 10 mg or 15 mg of the AA₁RA in a continuous infusion, and the like.

In some embodiments, the AA₁RA, e.g., KW-3902, can be provided in a continuous infusion for a period of time of about 2 hours, about 3 hours, about 4 hours, about 5 hours, about 6 hours, about 7 hours, about 8 hours, about 9 hours, about 10 hours, about I 1 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or more. Preferably, the AA₁RA, e.g., KW-3902 or a pharmaceutically acceptable salt, ester, amide, metabolite, or prodrug thereof can be provided in a continuous infusion for a period of time of about 3 hours, 3.5 hours, 4 hours, 4.5 hours, or 5 hours, 5.5 hours, 6 hours, or 6.5 hours, or any amount of time in between.

In some embodiments, a single dose of the an AA₁RA, e.g., KW-3902, is provided in a continuous infusion over a period of about 3 hours, 3.5 hours, 4 hours or 4.5 hours, preferably about 4 hours. In some embodiments, two doses of the AA₁RA, e.g., KW-3902 or a pharmaceutically acceptable salt, ester, amide, metabolite, or prodrug thereof can be provided in a continuous infusion over a period of about 4 hours, 4.5 hours, 5 hours, 5.5 hours, 6 hours, 6.5 hours, or 7 hours. Optionally, the first dose can be provided over about 1.5 to about 2.5 hours, preferably 2 hours, and the second dose can be provided over about 3.5 hours, 4 hours, or 4.5 hours, preferably about 4 hours.

In some embodiments, the patient is administered doses of a therapeutically effective amount of AA₁RA about every four days to about every month, e.g., an “effective periodic dose.” Accordingly, in some embodiments, the AA₁RA can be administered to the individual at least about every 4 days, about every 5 days, about every 6 days, about every 7 days, about every 8 days, about every 9 days, about every 10 days, about every 11 days, about every 12 days, about every 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 31 days, about every 40 days, about every 50 days, or about every 60 days, or any number of days in between. For example, in some embodiments, the patient is administered the AA1RA on a bi-weekly to bi-monthly basis. In some embodiments, the AA₁RA, e.g., KW-3902 is administered daily, twice a day, three times a day, four times a day, five times a day, six times a day, or more.

As stated above, the AA₁RA, e.g., KW-3902 or pharmaceutically acceptable salts, ester, amides, metabolites or prodrugs thereof can be administered orally. Optionally, the oral formulation of KW-3902 can provide for controlled release or sustained release of the active pharmaceutical ingredient, e.g., KW-302. Oral formulations of KW-3902 including controlled release formulation can be generated using routine methods known to those of skill in the art. A description of carrier materials useful in the oral formulations described herein can be found in the Remington: The Science and Practice of Pharmacy (20^{th} ed, Lippincott Williams & Wilkens Publishers (2003)), which is incorporated herein by reference in its entirety.

In some embodiments, wherein the patient receives at least one other therapeutic in addition to the AA1RA, patient can be administered said non adenosine-modifying diuretic, or other therapeutic (e.g., ACE, ARB, beta blocker, an aldosterone inhibitor and the like) and said an AA₁RA, e.g., KW-3902, nearly simultaneously. These embodiments include those in which the AA₁RA, e.g., KW-3902 and the non adenosine-modifying diuretic, or other therapeutic (e.g., ACE, ARB, beta blocker, an aldosterone inhibitor and the like) are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e,, one pill is taken right after the other or that one injection of one compound is made right after the injection of another compound, etc.

In other embodiments the administering step comprises administering non AA₁RA therapeutic, (e.g., ACE, ARB, beta blocker, an aldosterone inhibitor and the like) first and then administering an AA₁RA, erg., KW-3902. In yet other embodiments, the administering step comprises administering an AA₁RA, e.g., KW-3902 first, and then administering the non adenosine-modifying diuretic, or other therapeutic (e.g., ACE, ARB, beta blocker, an aldosterone inhibitor and the like). In these embodiments, the subject may be administered a composition comprising one of the compounds and then at some time, a few minutes or a few hours, later be administered another composition comprising the other one of the compounds. Also included in these embodiments are those in which the subject is administered a composition comprising one of the compounds on a routine or continuous basis while receiving a composition comprising the other compound occasionally.

Doses of the diuretic used in the methods described herein can be determined by conventional methods known to those skilled in the art. It will be appreciated by those skilled in the art that dosage amount and interval of the diuretic therapy may be adjusted individually to provide plasma levels of the active moiety that are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

In certain embodiments, the non adenosine-modifying diuretic used in the methods of the present invention is furosemide. In some embodiments, furosemide is administered in a dose of 20 mg, 40 mg, 60 mg, 80 mg, 100 mg, 120 mg, 140 mg, or 160 mg, or higher. The administration may be oral or intravenous. When furosemide is administered intravenously, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

The following example shows the concentration of KW-3902 and the M1 trans metabolite in individuals.

### Example 1

### Pharmacokinetic Study of KW-3902

Individuals received a single intravenous dose of 1 mg, 2.5 mg. 5 mg, 10 mg, 20 mg, 30 mg 40 mg, 50 mg or 60 mg KW-3902 as indicated. The serum concentrations of KW-3902 and its M1-trans metabolite were measured using routine analytical methods. Routine mathematical formulae were used to determine pharmacokinetic values. The data are presented in Table 1.

TABLE 1

| | | Group 1 | | Group 2 | | |
|---|---|---|---|---|---|---|
| | | 1 mg | 2.5 mg | 2.5 mg | 5 mg | 10 mg |
| Parameter | Statistic | (n = 5) | (n = 4) | (n = 8) | (n = 6) | (n = 6) |
| Cₘₐₓ | Mean | 12.9 | 35.6 | 35.9 | 90.9 | 186 |
| (ng/mL) | Min, Max | 10.8, 16.9 | 32.1, 43.9 | 24.4; 46.3 | 62.3; 141 | 156; 218 |
| | CV | 19 | 16 | 18 | 30 | 14 |
| Tₘₐₓ | Median | 0.5 | 1.2 | 0.5 | 0.5 | 0.5 |
| (h) | Min, Max | 0.5, 1.2 | 1.2, 1.2 | 0.5; 1.2 | 0.5; 1.2 | 0.5; 1.2 |
| T_{1/2} | Mean | 2.8 | 11.4 | 7.3 | 9.6 | 14.4 |
| (h) | Min, Max | 1.0, 5.7 | 5.2, 16.5 | 3.2; 16.3 | 4.7; 19.2 | 10.3; 20.8 |
| | CV | 82 | 43 | 59 | 63 | 27 |
| AUC_{∞} | Mean | 31.5 | 97.6 | 84 | 221 | 496 |
| (ng*h/mL) | Min, Max | 19.7, 56.9 | 75.8, 131 | 69.7; 135 | 132; 390 | 380; 704 |
| | CV | 48 | 24 | 26 | 46 | 24 |
| MRT_{∞} | Mean | 2.6 | 8.1 | 4.7 | 5.8 | 8.7 |
| (h) | Min, Max | 1.1, 5.0 | 3.2, 12.6 | 1.7; 10.5 | 2.2; 12.5 | 6.3; 15.7 |
| | CV | 75 | 51 | 61 | 75 | 41 |
| CL_{∞} | Mean | 36.6 | 26.6 | 31.1 | 26.1 | 21 |
| (L/h) | Min, Max | 17.6, 50.8 | 19.1, 33.0 | 18.6; 35.9 | 12.8; 38.0 | 14.2; 26.3 |
| | CV | 36 | 21 | 19 | 36 | 21 |
| Vss_{∞} | Mean | 58.9 | 185 | 117 | 106 | 161 |
| (L) | Min, Max | 27.9, 123 | 87.8, 259 | 42.1; 186 | 48.6; 157 | 135; 215 |
| | CV | 70 | 41 | 43 | 43 | 18 |

| | | Group 3 | | Group 4 | | |
|---|---|---|---|---|---|---|
| | | 20 mg | 30 mg | 40 mg | 50 mg | 60 mg |
| Parameter | Statistic | (n = 6) | (n = 6) | (n = 5) | (n = 6) | (n = 6) |
| Cₘₐₓ | Mean | 294 | 457 | 452 | 579 | 682 |
| (ng/mL) | Min, Max | 219, 366 | 300, 709 | 388; 568 | 426; 790 | 575; 777 |
| | CV | 20 | 30 | 17 | 25 | 12 |
| Tₘₐₓ | Median | 1.2 | 0.6 | 2.2 | 1.6 | 2.2 |
| (h) | Min, Max | 0.5, 1.2 | 0.5, 1.2 | 1.0; 2.2 | 1.0; 2.2 | 1.0; 2.2 |
| T_{1/2} | Mean | 18 | 14.7 | 16.1 | 15.5 | 16.4 |
| (h) | Min, Max | 12.1, 22.6 | 11.9, 16.7 | 12.5; 18.6 | 13.7; 19.0 | 10.4; 20.8 |
| | CV | 22 | 13 | 17 | 13 | 24 |
| AUC_{∞} | Mean | 898 | 1193 | 1871 | 2122 | 2724 |
| (ng*h/mL) | Min, Max | 678, 1157 | 709, 1822 | 1424; 2281 | 1313; 3272 | 2102; 3363 |
| | CV | 24 | 31 | 22 | 32 | 19 |
| MRT_{∞} | Mean | 11.5 | 7.7 | 9.4 | 7.9 | 9.2 |
| (h) | Min, Max | 7.9, 17.1 | 5.3, 8.7 | 6.3; 12.1 | 5.9; 12.3 | 4.8; 13.6 |
| | CV | 32 | 17 | 25 | 30 | 35 |
| CL_{∞} | Mean | 23.4 | 27.3 | 22.3 | 25.6 | 22.7 |
| (L/h) | Min, Max | 17.3, 29.5 | 16.5, 42.3 | 17.5; 28.1 | 15.3; 38.1 | 17.8; 28.5 |
| | CV | 24 | 32 | 23 | 31 | 20 |
| Vss_{∞} | Mean | 244 | 189 | 178 | 166 | 176 |
| (L) | Min, Max | 201, 316 | 133, 222 | 149; 208 | 121; 202 | 109; 225 |
| | CV | 18 | 19 | 15 | 18 | 27 |

The mean value for T_{1/2} of a 30 mg dose of KW-3902 was 14.7 hours.

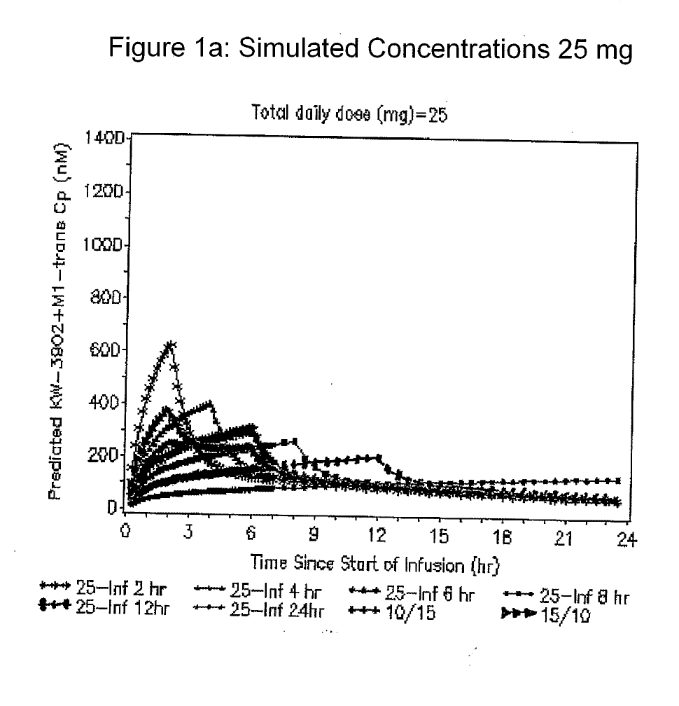

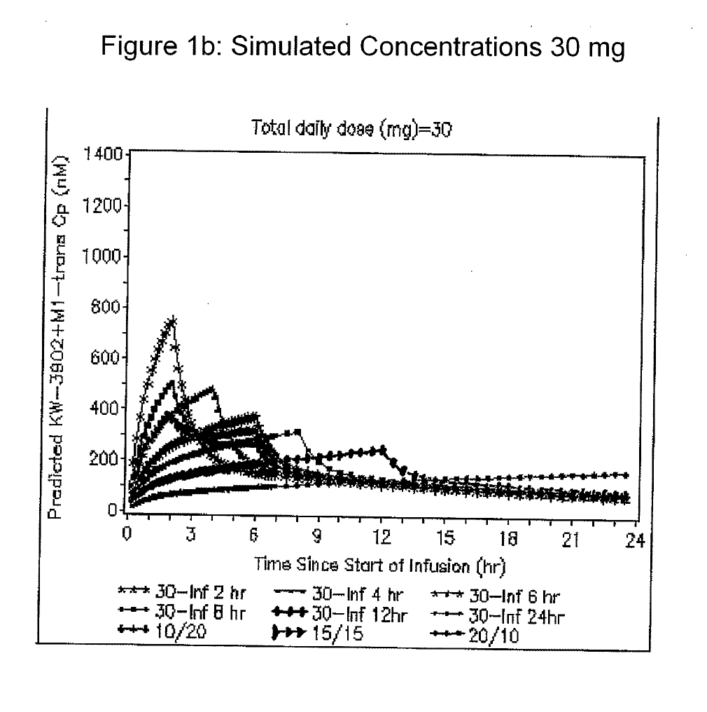

These data were extrapolated to generate the curves showing simulated serum concentrations of KW-3902 and M1-trans following intravenous infusions of KW-3902. The curves of the simulated concentrations are shown in FIGS. 1A and 1B. Briefly, both 1A and 1B show the predicted serum concentrations of KW-3902 and M1-trans over time following single 25 mg or 30 mg intravenous doses of KW-3902. The curves show the predicted concentrations with 2, 4, 6, 8, 12, or 24 hours infusion times, as indicated in the figure legend. FIG. 1A also includes the simulated serum concentration of KW-3902 and M1-trans following a 10 mg dose of KW-3902 administered over two hours, followed by a 15 mg dose of KW-3902 over 4 hours (10/15), or a 15 mg dose of KW-3902 over two hours, followed by a 10 mg dose of KW-3902 over 4 hours (15/10). FIG. 1B also shows the simulated serum concentration of KW-3902 and M1-trans following a 10 mg dose of KW-3902 over 2 hours followed by a 20 mg dose of KW-3 902 over 4 hours (10/20); a 15 mg dose of KW-3902 administered over 2 hours followed by a 15 mg dose of KW-3902 over 4 hours (15/15), or a 20 mg dose of KW-3902 administered over 2 hours followed by a 10 mg dose of KW-3902 over 4 hours (20/10). The arrows in FIG. 1B indicate the simulated serfu concentration of KW-3902 and M1-trans following a single dose of 25 mg or 30 mg KW-3902 administered over the course of 4 hours.

The following examples demonstrate that KW-3902 has persistent beneficial effects on renal function after it has effectively been cleared from the patients' body.

### EXAMPLE 2

### Treatment of Individuals with Stable CHF

A double-blind, multi-center, cross-over designed, controlled study was conducted as follows: Approximately 23 outpatient subjects were randomized. Subjects had an estimated creatinine clearance between 30 mL/min and 80 mL/min., and were taking ≧80 mg furosemide daily. Subjects received at least two treatments that were at least three days apart, with a median interval between treatments of 6 days. Each patient received either 30 mg KW-3902 IV or placebo over 120 min, in addition to 80 mg IV furosemide over 120 min. Patients that received KW-3902 on the first visit received placebo on the second visit, and vice versa. Infusions of lothalamate (CONRAY™) and para-aminohippurate were administered according to standard protocols to assess glomerular filtration rate (GFR) and renal plasma flow (RPF), respectively. Iothalamate and para-aminohippurate were administered over 180 minutes before treatment with KW-3902/placebo and 8 hours after treatment with KW-3902/placebo. Urine and plasma were collected for GFR and RPF at baseline and 2, 4, and 8 hours. The mean GFR at baseline was 61 mL/min.

The data are presented in Table 2, below.

TABLE 2

| | 0-2 hours | 2-4 hours | 4-8 hours | 0-8 hours |
|---|---|---|---|---|
| | % Difference Between KW-3902 and Placebo | | | |
| GFR | 27.3* | 7.6 | 30.1* | 23.4* |
| RPF | 27.7* | 12.7 | 30.0* | 24.9* |

Percent Change from Baseline

GFR

| | | | | |
|---|---|---|---|---|
| KW-3902 | 22.1* | 8.5 | 41.3* | 32.1* |
| Placebo | −0.6 | 3.7 | 12.3 | 8.3 |

RPF

| | | | | |
|---|---|---|---|---|
| KW-3902 | 33.3* | 41.7 | 55.8* | 47.7** |
| Placebo | 3.7 | 18.2 | 17.8 | 16 |

*p < 0.05
**p < 0.01

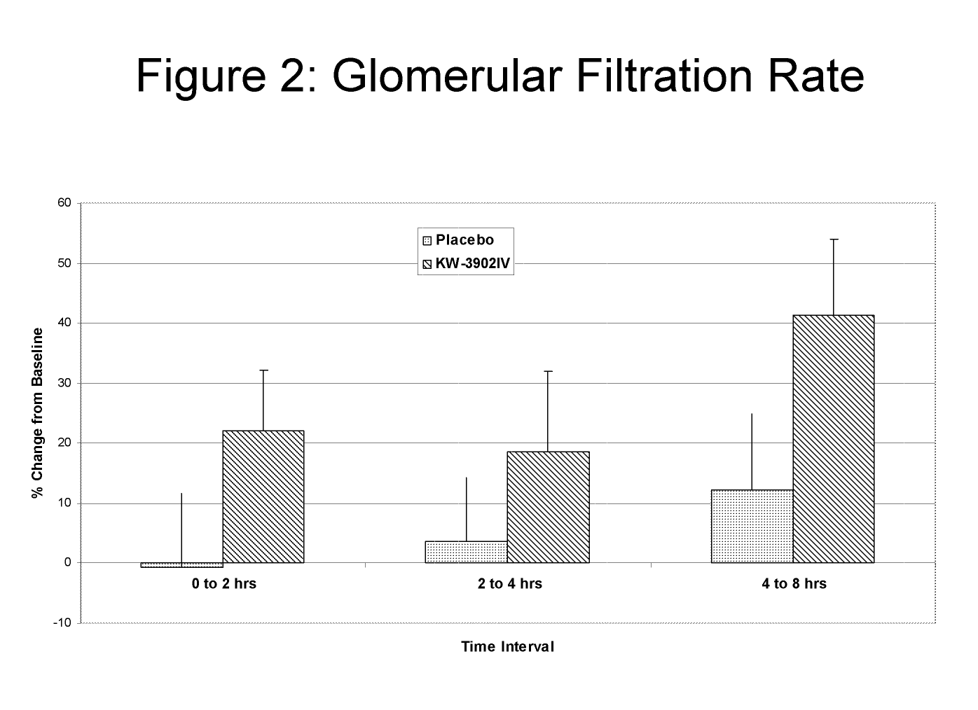

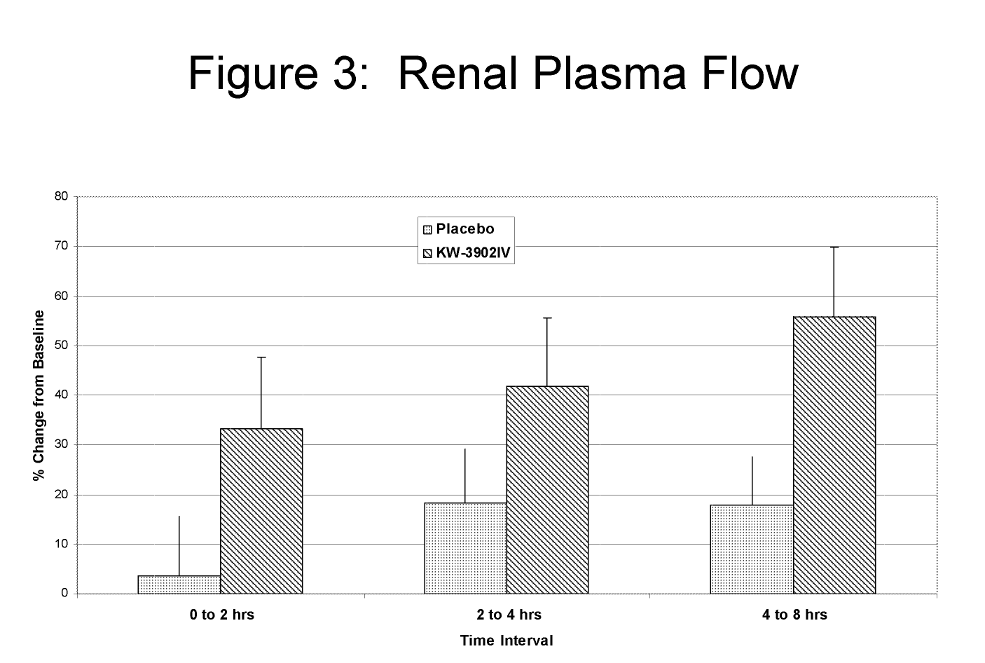

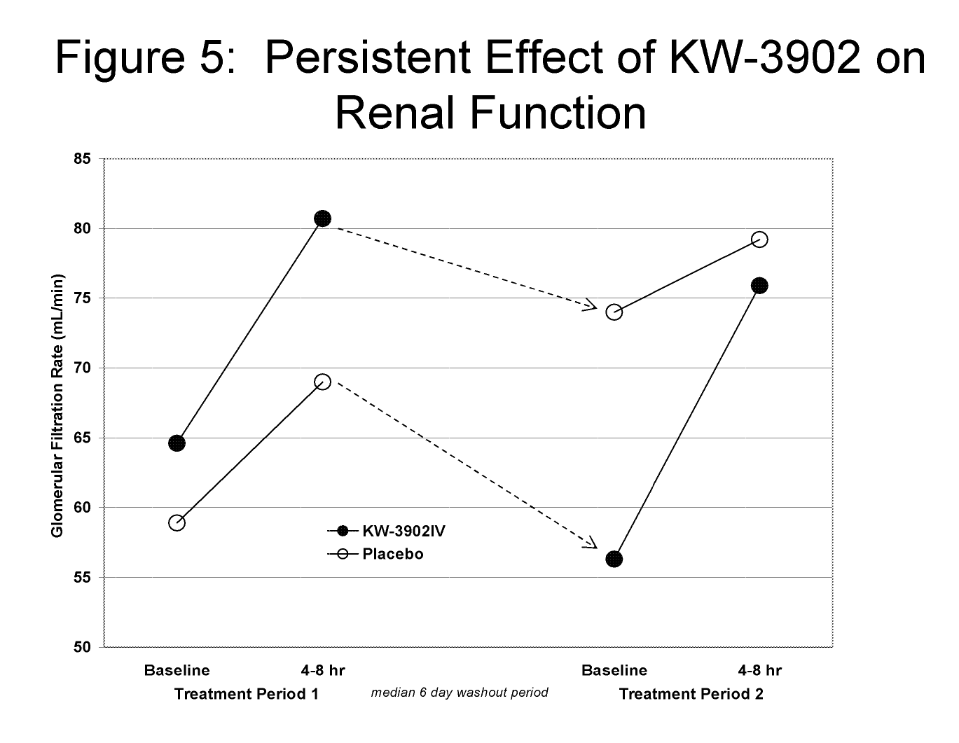

Urine volume over 8 hours was greater during the KW-3902 plus furosemide (2995.1±1138.17 mL) than placebo plus furosemide (2535.7±1159.35 mL). Unexpectedly, in the subgroup that received KW-3902 on the first visit, the increase in GFR notes at 8 hours persisted at the time of follow-up baseline (median 6 days). FIG. 5. The results demonstrate that the AA1RA KW-3902 produces a statistically significant increase in GFR (FIG. 2) and renal plasma flow (FIG. 3) in heart failure patients with renal impairment who are treated with diuretics. As shown in FIG. 5, this improvement in renal fimetion unexpectedly persists for days, i.e., long after KW-3902 and its metabolites remain in the patients' system. The data demonstrate that AA1RAs provides significant benefits in individuals, in particular in whom renal impairment limits heart failure therapy.

### EXAMPLE 3

### KW-3902 Improves Renal Function in CHF Patients Refractory to Standard Diuretic Therapy

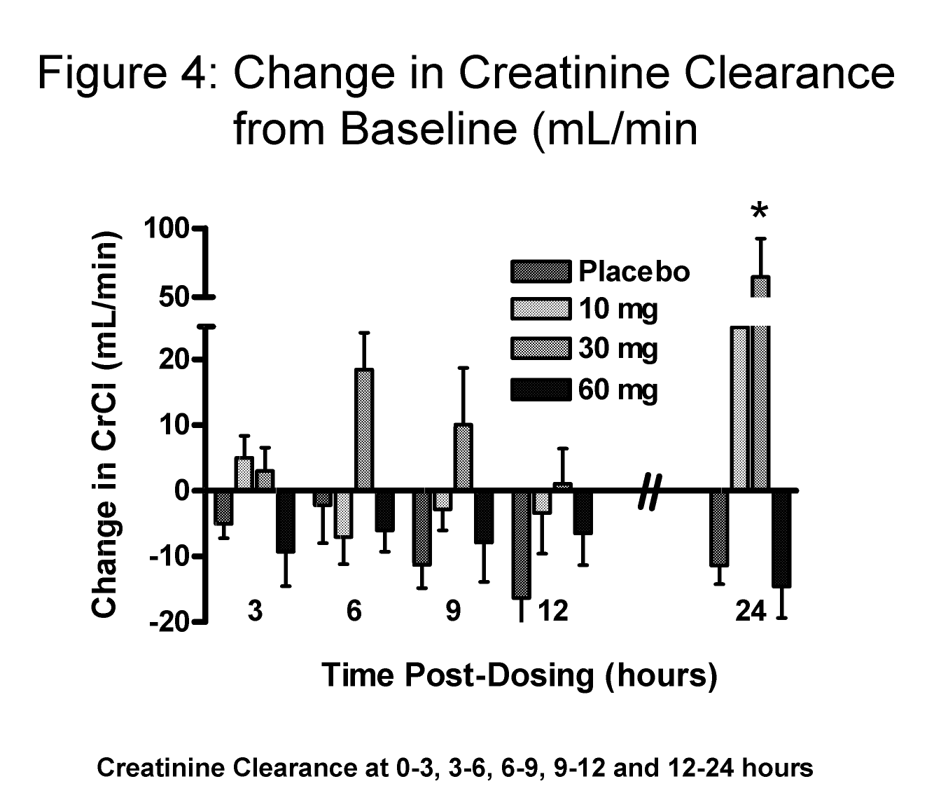

A double-blind placebo-controlled study was conducted as follows: 35 subjects with congestive heart failure that were refractory to standard diuretic therapy were identified. The subjects were randomized and divided into four treatment groups: (1) Placebo (n=11); (2) KW-3902, 10 mg (n=8); (3) KW-3902, 30 mg (n=7); and (4) KW-3902, 60 mg (n=7). The patients' diuretic therapy was stopped at least 5 hours before prior to the treatment. Staring at −3 hours, urine was collected for volume and creatinine clearance measurements. At 0 hours, the patients were given an intravenous infusion of KW-3902 or placebo in combination with the patients' current diuretic therapy. Blood and urine samples were colleted at 0, 3, 6, 9, 12 and 24 hours following study drug administration. The change in creatinine clearance from baseline (mL/min) in the various treatment groups at 0-3 hours, 3-6 hours, 6-9 hours, 9-12, and 12-24 hours is shown in FIG. 4. The data demonstrate that a dose of 30 mg KW-3902 resulted in an improvement in renal function over time.

### EXAMPLE 4

### KW 3902 Improves Renal Function in CHF Patients with Acute Fluid Overload and Renal Impairment

A double-blind, randomized multi-center, placebo controlled study was conducted as follows: Approximately 157 subjects were randomized to yield 144 evaluable subjects in an intent-to treat analysis conducted at approximately 50 sites. The study population included males and females at least 18 years of age with New York Heart Association Class II-IV CHF. All subjects had an estimated creatinine clearance between 20 mL/min and 80 mL/min. The average serum creatinine for all individuals at entry was 1.75 mg/dL. All subjects were taking an oral loop diuretic. The demographic data for the study is presented in Table 3 below.

TABLE 3
STUDY DEMOGRAPHICS

| | KW-3902 | | | | |
|---|---|---|---|---|---|
| | Placebo | 2.5 mg | 15 mg | 30 mg | 60 mg |
| n = (ITT population) | 27 | 29 | 30 | 29 | 29 |
| Age (mean yrs) | 67 | 64 | 69 | 66 | 67 |
| Sex (% M/% F) | 74/26 | 66/34 | 65/35 | 70/30 | 69/31 |
| NYHA Class II (%) | 4 | 0 | 0 | 3 | 7 |
| NYHA Class III (%) | 52 | 41 | 58 | 47 | 52 |
| NYHA Class IV (%) | 44 | 59 | 42 | 50 | 41 |

Study visits included pre-treatment days −2 to −1, days 1 to 3 of the Treatment Period, day 4/early Termination and a follow up contact at day 30. Procedures and observations included medical history, physical examination, classification of CHF, vital signs, body weight, CHF signs and symptom scores, Holter monitor recording, chest X-ray, CBC chemistries, creatinine clearance, fluid intake, and urine output.

On treatment days, individuals received KW-3902 intravenously over 120 minutes at one of four doses (2.5 mg, 15 mg, 30 mg, or 60 mg) vs. placebo as both monotherapy and concomitant therapy with diuretics. KW-3902 (or placebo) was administered on days 1 through 3. On day 1, KW-3902 (or placebo) was administered as a monotherapy. 6 hours after administration of KW-3902, IV loop diuretic was given to all treatment groups as needed. On days 2 and 3 KW-3902 was administered as combination therapy with intravenous furosemide, if clinically indicated. Final laboratory data were collected on day 4 or early termination. A follow-up phone contact was conducted on day 30.

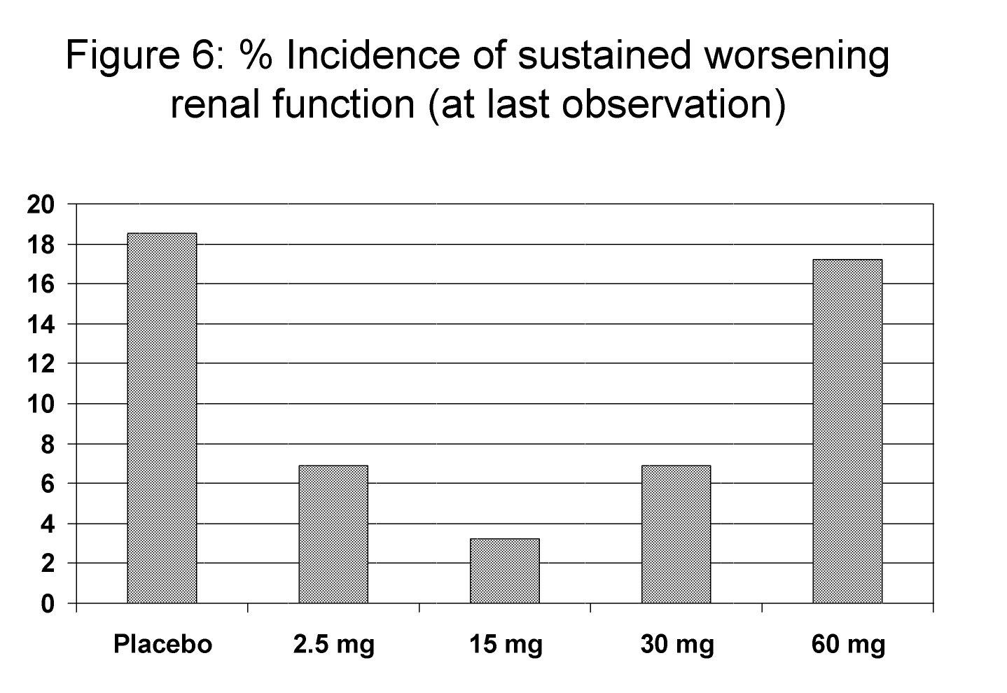

The percentage of subjects presenting with sustained worsening renal function was determined. Worsening renal function was defined as a ≧0.3 mg/dL increase in serum creatinine. The results are presented in FIG. 6. The results demonstrate that KW-3902 improves renal function in patients with CHF and renal impairment. The beneficial effect on renal function was persistent.

### EXAMPLE 4B

### KW 3902 Improves Renal Function in CHF Patients with Acute Fluid Overload and Renal Impairment

More than 300 subjects hospitalized due to acute CHF requiring intravenous diuretic therapy to treat fluid overload, and presenting with creatinine clearance values between 20 to 80 mL/min were identified. The subjects were randomized to receive either placebo, or 10 mg, 20 mg or 30 mg intravenous KW-3902 per day. The subjects' BNP levels, NT-Pro-BNP, serum creatinine, weight, and NYHA Class were measured.

On day 1, KW-3902 (or placebo) was co-administered with intravenous furosemide (LASIX™). The specified dose of KW-3902 (or placebo) was infused over a four hour time period. Subjects received therapy for up to three days, as required. Serum creatinine, weight, dyspnea, and worsening heart failure were assessed daily by the investigator on days 1 through 14. Worsening renal function was defined as a ≧0.3 mg/dL increase in serum creatinine.

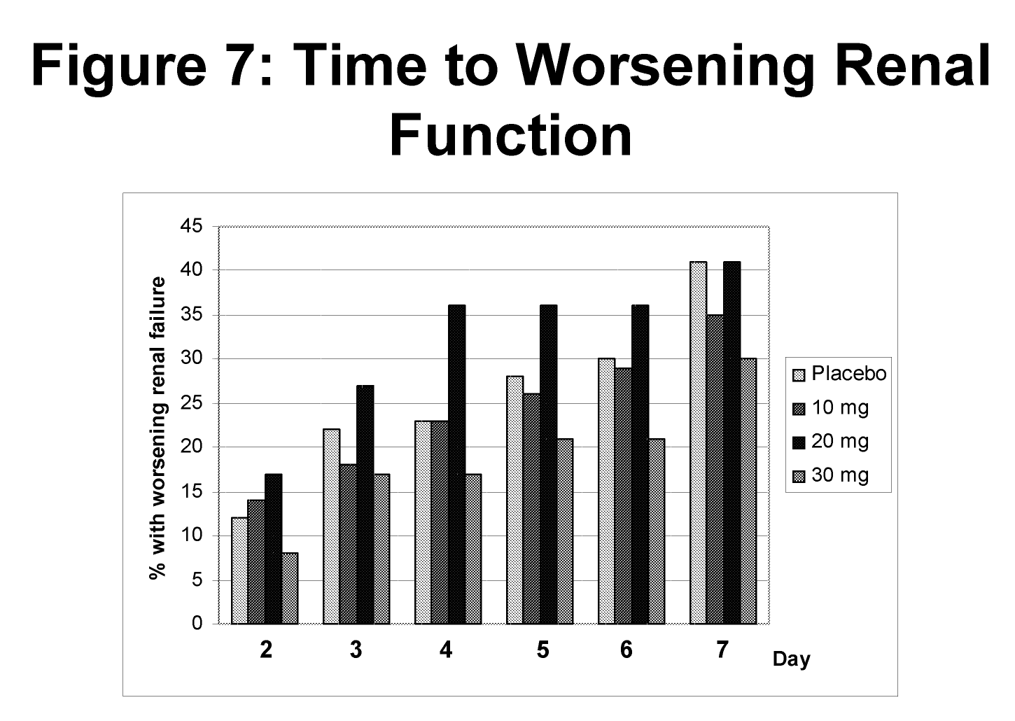

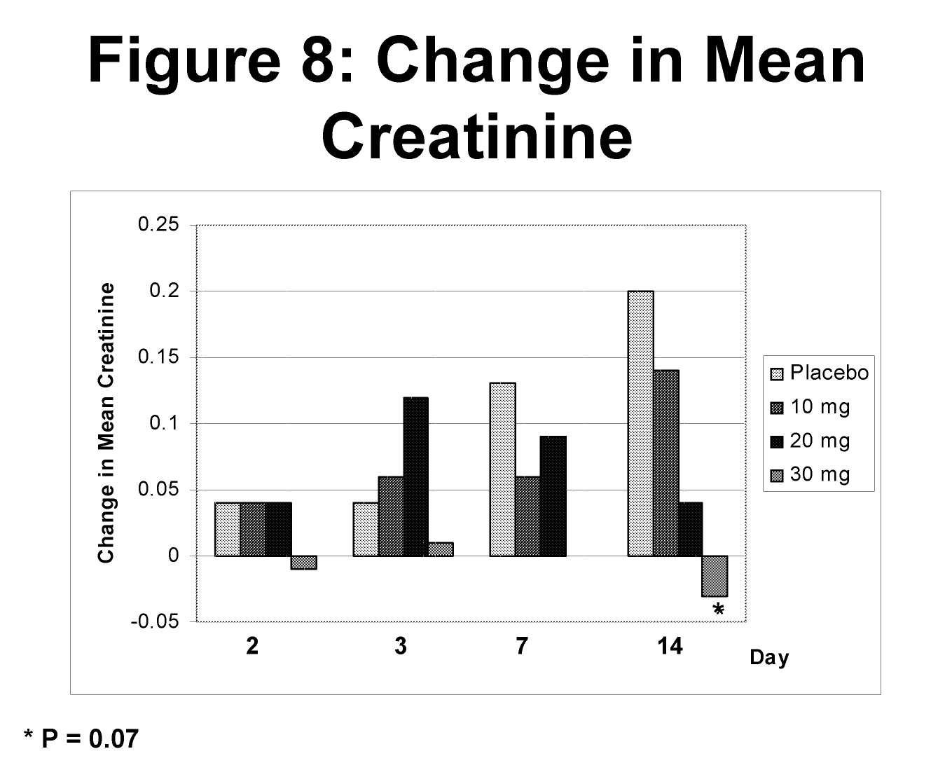

The data from the study are presented in FIGS. 7 and 8. Subjects receiving placebo exhibited a greater increase in serum creatinine levels over time. Notably, subjects that received 30 mg KW-3902 showed an overall decrease in serum creatinine levels, indicative of an improvement in renal function at Day 14, whereas subjects that received placebo showed a mean increase in serum creatinine levels at Day 14, indicative of worsening renal function (FIG. 8). Notably, though KW-3902 was only administered over Days 1, 2, and 3, the improvement in serum creatinine levels was observed on Day 14, demonstrating that KW-3902 has a persistent effect on renal function. Furthermore, the increase in the percentage of individuals with worsening renal failure on days 2, 3, 4, 5, 6, and 7 of the study was lower in the treatment group receiving KW-3902 therapy compared to the placebo treatment group. FIG. 7. These data confirm that KW-3902 improves and maintains renal function.

A subgroup of subjects received a single dose of KW-3902 and LASIX or a single dose of placebo and LASIX. The serum creatinine levels at Day 1 and Day 14, and the change in serum creatinine levels is presented in Table 4, below.

TABLE 4

| | Day 1 Creatinine | Day 14 Creatinine | Δ Creatinine |
|---|---|---|---|
| Treatment | (mg/dL) | (mg/dL) | (mg/dL) |
| Placebo | 2.4 | 3 | 0.6 |
| Placebo | 1.5 | 1.0 | −0.5 |
| Placebo | 1.4 | 1.6 | 0.2 |
| Placebo | 1.3 | 1.7 | 0.4 |
| KW-3902 | 1.0 | 1.0 | 0 |
| KW-3902 | 1.7 | 1.2 | −0.5 |
| KW-3902 | 1.4 | 1.4 | 0 |
| KW-3902 | 1.15 | 1.3 | 0.2 |
| KW-3902 | 2.9 | 2.7 | −0.2 |
| KW-3902 | 1.6 | 2.7 | −0.1 |
| KW-3902 | 3.2 | n/a | n/a |

This beneficial effect is seen long after KW-3902 is projected to be cleared from the patients' system. In the placebo group, there was a mean increase in serum creatinine levels at Day 14, while in the treatment group receiving AA1RA therapy (30 mg KW-3902), there was a mean decrease in serum creatinine levels. These data demonstrate an improvement in renal function after a single dose of KW-3902. This improvement persists over a time period well beyond that which KW-3902 is expected to be present in the patients' system.

What is claimed is:

1. A method for treating a patient, comprising:
providing a therapeutically-effective amount of an AA₁RA to the patient; and
informing the patient or a medical care worker that administration of the AA₁RA can provide an improvement in renal function that persists for a time period following administration of the AA₁RA, and wherein the time period is at least about 3 days.

2. The method of claim 1, wherein said time period is at least about 5 days.

3. The method of claim 1, wherein said time period is at least about 7 days.

4. The method of claim 1, wherein said time period is at least about 10 days.

5. The method of claim 1, wherein said time period is at least about 14 days.

6. The method of claim 1, wherein the patient is suffering from congestive heart failure.

7. The method of claim 1, wherein said AA₁RA is selected from the group consisting of KW-3902, BG-9719, BG-9928, CVT-124, and SLV-320, or a pharmaceutically acceptable salt, ester, amide, prodrug, or metabolite thereof.

8. The method of Claim I, wherein said AA₁RA is KW-3902 or a pharmaceutically acceptable salt, ester, amide, prodrug, or metabolite thereof.

9. The method of claim 1, wherein said therapeutically effective amount of KW-3902 is between about 2.5 to about 70 mg/dose.

10. The method of claim 9, wherein said therapeutically effective amount of KW-3902 is between about 20 mg/dose to about 50 mg/dose.

11. The method of claim 10, wherein said therapeutically effective amount of KW-3902 is about 30 mg/dose.

12. A method for treating a patient, comprising:
identifying a patient in need of a relatively long term improvement in renal function; and
administering a relatively shorter term AA₁RA therapy to the patient.

13. The method of claim 12, wherein the relatively long term improvement is an improvement that persists at least about 3 days after the AA₁RA therapy.

14. The method of claim 12, wherein said improvement persists at least about 5 days after the AA₁RA therapy.

15. The method of claim 12, wherein said improvement persists at least about 7 days after the AA₁RA therapy.

16. The method of claim 12, wherein said improvement persists at least about 10 days after the AA₁RA therapy.

17. The method of claim 12, wherein said improvement persists at least about 14 days after the AA₁RA therapy.

18. The method of claim 12, wherein the patient is suffering from congestive heart failure.

19. The method of claim 12, wherein said AA₁RA is selected from the group consisting of KW-3902, BG-9719, BG-9928, CVT-124, and SLV-320, or a pharmaceutically acceptable salt, ester, amide, prodrug, or metabolite thereof.

20. The method of claim 12, wherein said AA₁RA is KW-3902 or a pharmaceutically acceptable salt, ester, amide, prodrug, or metabolite thereof.

21. The method of claim 20, wherein said therapeutically effective amount of KW-3902 is between about 2.5 to about 70 mg/dose.

22. The method of claim 21, wherein said therapeutically effective amount of KW-3902 is between about 20 mg/dose to about 50 mg/dose.

23. The method of claim 22, wherein said therapeutically effective amount of KW-3902 is about 30 mg/dose.

24. The method of claim 12, further comprising informing the patient or a medical care worker that improvement in renal function persists for a time period following administration of the AA₁RA, and wherein the time period is at least about 3 days.

25. The method of claim 12, wherein the patient suffers from congestive heart failure.

26. A method for treating a patient having impaired renal function, comprising:
designating a course of therapy that is intended to achieve improved renal function for at least a predetermined period, wherein the course of therapy includes administration of an AA₁RA; and
administering the course of therapy to the patient, wherein the AA₁RA administration is completed at least about 3 days prior to completion of the predetermined period.

27. A method of improving and/or maintaining renal function in individuals with stable congestive heart failure (CHF) taking chronic diuretics, comprising
administering to said individual a therapeutically effective amount of an adenosine At receptor antagonist (AA₁RA) in about four day to about monthly intervals, wherein said individual simultaneously continues said chronic diuretic therapy throughout the course of treatment with said AA₁RA.

28. The method of claim 27, wherein said AA₁RA is selected from the group consisting of KW-3902, BG-9719, BG-9928, CVT-124, and SLV-320, or a pharmaceutically acceptable salt, ester, amide, prodrug, or metabolite thereof.

29. The method of claim 27, wherein said AA₁RA is KW-3902 or a pharmaceutically acceptable salt, ester, amide, prodrug, or metabolite thereof.

30. The method of claim 29, wherein said therapeutically effective amount of KW-3902 is between about 2.5 to about 70 mg/dose.

31. The method of claim 30, wherein said therapeutically effective amount of KW-3902 is between about 20 mg/dose to about 50 mg/dose.

32. The method of claim 31, wherein said therapeutically effective amount of KW-3902 is about 30 mg/dose.

33. The method of claim 27, wherein said AA₁RA is administered in approximately 4 to 14 day intervals.

34. The method of claim 27, wherein said AA₁RA is administered in approximately 7 to 9 day intervals.

35. The method of claim 27, wherein said AA₁RA is administered in approximately 7 to 30 day intervals.

36. The method of claim 27, wherein said AA₁RA is administered in approximately 14 day intervals.

37. A method of treating an individual experiencing mild renal impairment, wherein said individual is undergoing diuretic therapy comprising
administering to said individual a therapeutically effective amount of an AA₁RA on a bi-weekly to monthly basis.

38. The method of claim 37, wherein said AA₁RA is selected from the group consisting of KW-3902, BG-9719, BG-9928, CVT-124, and SLV-320, or a pharmaceutically acceptable salt, ester, amide, prodrug, or metabolite thereof.

39. The method of claim 38, wherein said AA₁RA is KW-3902 or a pharmaceutically acceptable salt, ester, amide, prodrug, or metabolite thereof.

40. The method of claim 39, wherein said therapeutically effective amount of KW-3902 is between about 2.5 to about 70 mg/dose.

41. The method of claim 40, wherein said therapeutically effective amount of KW-3902 is between about 20 mg/dose to about 50 mg/dose.

42. The method of claim 41, wherein said therapeutically effective amount of KW-3902 is about 30 mg/dose.

43. The method of claim 37, wherein said AA₁RA is administered in approximately 4 to 14 day intervals.

44. The method of claim 37, wherein said AA₁RA is administered in approximately 7 to 9 day intervals.

45. The method of claim 37, wherein said AA₁RA is administered in approximately 7 to 30 day intervals.

46. The method of claim 37, wherein said AA₁RA is administered in approximately 14 day intervals.

47. A method for slowing or reversing an existing or developing renal impairment in a patient, comprising
administering to the patient an effective periodic dose of an AA₁RA between about once every four days and about once every month.

48. The method of claim 47, wherein said AA₁RA is selected from the group consisting of KW-3902, BG-9719, BG-9928, CVT-124, and SLV-320, or a pharmaceutically acceptable salt, ester, amide, prodrug, or metabolite thereof.

49. The method of claim 48, wherein said AA₁RA is KW-3902 or a pharmaceutically acceptable salt, ester, amide, prodrug, or metabolite thereof.

50. The method of claim 49, wherein said therapeutically effective amount of KW-3902 is between about 2.5 to about 70 mg/dose.

51. The method of claim 51, wherein said therapeutically effective amount of KW-3902 is between about 20 mg/dose to about 50 mg/dose.

52. The method of claim 52, wherein said therapeutically effective amount of KW-3902 is about 30 mg/dose.

53. The method of claim 48, wherein said AA₁RA is administered in approximately 4 to 14 day intervals.

54. The method of claim 48, wherein said AA₁RA is administered in approximately 7 to 9 day intervals.

55. The method of claim 48, wherein said AA₁RA is administered in approximately 7 to 30 day intervals.

56. The method of claim 48, wherein said AA₁RA is administered in approximately 14 day intervals.

57. The method of claim 12, further comprising informing the patient or a medical care worker that improvement in renal function persists for a time period following administration of the AA₁RA, and wherein the time period is at least about 5 days.
